(19) United States
(12) Plant Patent Application Publication
(10) Pub. No.: US 2014/0245510 P1
(43) Pub. Date: Aug. 28, 2014

(54) Spathiphyllum plant named 'Jet200801 Tin'

(50) ;

(71) Applicant: Wim Brouwer, Nootdorp (NL)

(72) Inventor: Wim Brouwer, Nootdorp (NL)

(21) Appl. No.: 13/815,382

(22) Filed: Feb. 26, 2013

(51) Int. Cl. A01H 5/00

(52) U.S. Cl. PLT/263.1

(57) ABSTRACT

A new and distinct Spathiphyllum cultivar named ‘Jet200801 Tin’ is disclosed, characterized by large, dark green, foliage. Plants of the new variety are large and vigorous with large, clear white spathes. The new variety is commercially suitable for indoor plant production.

### LATIN NAME OF THE GENUS AND SPECIES

Spathiphyllum hybrid

### VARIETY DENOMINATION

‘Jet200801 Tin’

### BACKGROUND OF THE INVENTION

The new cultivar is a product of a planned breeding program. The new variety originated from a cross pollination, made by the inventor, of a proprietary unpatented seed parent and a proprietary, unpatented pollen parent, both Spathiphyllum hybrids. The crossing was performed at a commercial greenhouse in the Netherlands during September of 2008.

The new variety was discovered by the inventor, Wim Brouwer, a citizen of the Netherlands, in Feb. 10, 2010 in a group of seedlings resulting from the crossing. The new cultivar was found in a commercial greenhouse belonging to the inventor in Nootdorp, the Netherlands.

Asexual reproduction of the new cultivar ‘Jet200801 Tin’ was first performed in the Netherlands by tissue culture Nov. 14, 2010. ‘Jet200801 Tin’ has been reproduced by micro-propagation and has shown that the unique features of this cultivar are stable and reproduced true to type.

### SUMMARY OF THE INVENTION

The cultivar ‘Jet200801 Tin’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Jet200801 Tin.’ These characteristics in combination distinguish ‘Jet200801 Tin’ as a new and distinct Spathiphyllum cultivar:

1. Dark green foliage.

2. Large foliage.

3. Clear white spathes.

4. Durable and easy to grow plant

5. Large spathes.

### PARENTAL COMPARISON

Plants of the new cultivar ‘Jet200801 Tin’ cannot be compared to the seed parent as records of those characteristics were not kept. ‘Jet200801 Tin’ is similar to plants of the pollen parent in most characteristics, however, ‘Jet200801 Tin’ has larger, darker colored foliage. Additionally, plants of the new variety are larger and produce new flowers more easily.

### COMMERCIAL COMPARISON

‘Jet200801 Tin’ can be compared to compared to the commercial variety Spathiphyllum ‘Jetty’ U.S. Plant Pat. No. 9,957. Plants of ‘Jetty’ are similar to plants of ‘Jet200801 Tin’ in most horticultural characteristics, however, ‘Jet200801 Tin’ grows faster than ‘Jetty’ and produces much larger flowers that are more clear white. Additionally, plants of the new variety are larger than plants of ‘Jetty’.

### BRIEF DESCRIPTION OF THE PHOTOGRAPH

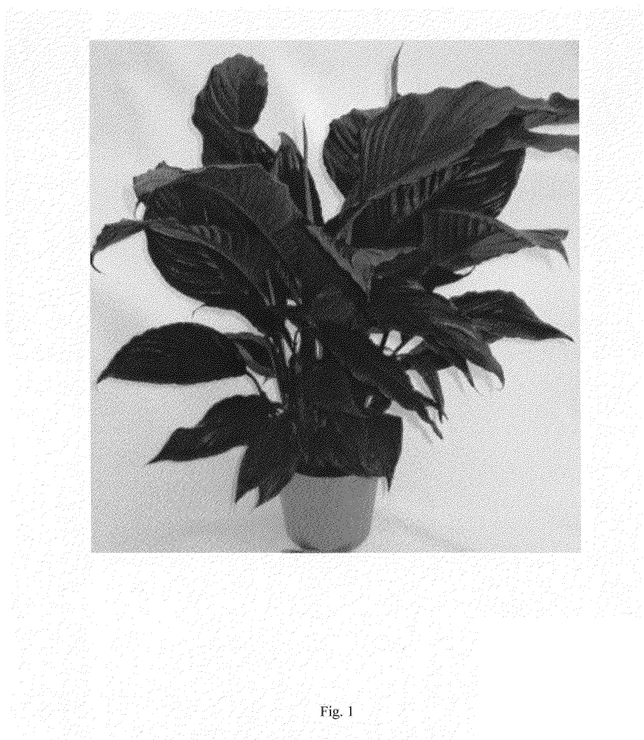

The accompanying photograph in FIG. 1 illustrates in full color a typical plant of ‘Jet200801 Tin’ grown in a greenhouse in Altha, Fla. This plant is approximately 6 months old, shown in a 6 inch pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001 except where general terms of ordinary dictionary significance are used. The following observations and measurements describe ‘Jet200801 Tin’ plants grown in a climate controlled greenhouse in Apopka, Fla., USA. Temperatures ranged from 18° C. to 22° C. at night to 18° C. to 22° C. during the day. No artificial light, photoperiodic treatments or chemical treatments were given to the plants. Measurements and numerical values represent averages of typical plant types.
Botanical classification: Spathiphyllum hybrid ‘Jet200801 Tin’
Propagation:
   Time to initiate roots.—Approximately 18 days at approximately 25° C. soil temperature.
   Time to produce a fully rooted plantlet.—Approximately 14 weeks at approximately 20 to 30° C. soil temperature.
   Root description.—Thick, fleshy roots with fine, fibrous lateral roots. Color near Orange-White 158C.
Plant:
   Growth habit.—Leaves growing directly from base of plant, with no stems.
   Plant shape.—Upright, leaves slightly arching downward.
   Height.—Approximately 85 cm.
   Plant spread.—Approximately 70 cm in a 6 inch pot.
   Growth rate.—Rapid.
   Branching characteristics.—No branching. Leaves emerge direct from base of plant.
   Number of clumps of leaves.—12.
   Number of leaves per clump.—Average 6.
   Number of leaves per plant.—Approximately 72.
   Age of plant described.—Approximately 6 months.
   Pot size of plant described.—6 inch.
Foliage:
   Leaf.—Arrangement: Single leaves emerging basally. Average Length (excluding petiole): Average 50.2 cm. Average Width: Average 16.9 cm. Shape of blade: Elliptic. Aspect: Slightly recurved. Apex: Apiculate Base: Attenuate Margin: Entire. Texture of top surface: Smooth, moderately glossy. Texture of bottom surface: Smooth, moderately glossy. Color: Young foliage upper side: Near R.H.S. Green139B Young foliage under side: Near R.H.S. Green 137A Mature foliage upper side: Near R.H.S. Green139A Mature foliage under side: Near R.H.S. Green137B
   Venation.—Type: Pinnate. Venation coloration upper side: Green 143B Venation coloration under side: Green 143C.
   Petiole(excluding Geniculum).—Length: Approximately 32 cm Width: At geniculum: Approximately 0.8 cm Above clump: Approximately 1.3 cm Color: Near RHS Green 139A Strength: Moderate.
   Geniculum.—Length: Approximately 5.5 cm Width: Approximately 0.7 cm Color: Near RHS Green 137B.
Inflorescence:
   Arrangement.—Spathes with spadices held above the foliage on erect peduncles arising from the petiole sheath. Flowering Habit: Continuous Quantity of spathes per plant: On average 3 to 6 fully open spathes in good condition. Natural Flowering season: Autumn to Winter Time to flowering: 6 months Fragrance: None. Self-cleaning or persistent: Persistent Flower Longevity: Flowers stay in good condition approximately 6 to 12 weeks on the plant.
   Spathe.—Aspect: Concave. Length: Average 21 cm. Width: Average 9.1 cm. Shape: Elliptic Margin: Entire Apex: Apiculate Base: Cuneate Color: Front when opening: Near RHS White N155A. Back when opening: Near RHS White N155A, with mid vein near Green 143C. Front when mature: Near RHS White N155A Back when mature: Near RHS White N155A, with mid vein near Green 143C. Fading to: No fading.
   Spadix.—Shape: Columnar, arising from the top of the peduncle. Tip: Obtuse Base: Obtuse Length: Approximately 10.1 cm Width: Approximately 2.2 cm Color: When opening: Near RHS Yellow-White 158D Mature: Near RHS Yellow-White 158C Quantity of flowers per spadix: Approximately 150 Spadix flower arrangement: Bisexual, rounded Spadix flower diameter: Approximately 0.25 cm Spadix flower depth: Approximately 0.15 cm.
   Peduncle.—Length: Approximately 85 cm Width: Approximately 1.1 cm Color: Near RHS Green 139B Strength: Moderate.
Reproductive organs:
   Ovary color.—Near RHS 155B.
   Stigma color.—Near RHS 155B.
   Anther color.—Near RHS 155A.
   Quantity of pollen.—Scant.
   Pollen color.—Near RHS 155A.
Other characteristics:
   Disease resistance.—Neither resistance nor susceptibility to diseases or pests has been observed in this variety.
   Drought tolerance and cold tolerance.—The new cultivar is a typical Spathiphyllum, cold tolerant to approximately 5 to 7° C. and does not tolerate drought.
   Fruit/seed production.—Not observed.

What is claimed is:

1. A new and distinct cultivar of Spathiphyllum plant named ‘Jet200801 Tin’ as herein illustrated and described.
